(19) United States
(12) Patent Application Publication
(10) Pub. No.: US 2012/0295345 A1
(43) Pub. Date: Nov. 22, 2012

(54) APPARATUSES AND RELATED METHODS FOR DELIVERING BIOLOGICAL MATERIAL INTO A CELL

(76) Inventors: Quentin T. Aten, Orem, UT (US); Sandra H. Burnett, Saratoga Springs, UT (US); Brian D. Jensen, Orem, UT (US); Larry L. Howell, Orem, UT (US)

(21) Appl. No.: 13/475,401

(22) Filed: May 18, 2012

Related U.S. Application Data
(60) Provisional application No. 61/487,535, filed on May 18, 2011.

(51) Int. Cl. C12N 5/071; C12M 1/00; C12N 5/07; C12N 5/04; C12N 1/14; C12N 1/16

(52) U.S. Cl. 435/348; 435/254.1; 435/255.1; 435/349; 435/410; 435/375; 435/283.1

(57) ABSTRACT

Systems, devices, and methods for protecting a biological material during delivery into a biological structure are provided. In one aspect, for example, a device for protecting and delivering a preselected biological material into a biological structure can include a lance operable to maintain a charge capable of associating a biological material thereto and at least one protective region formed on or in the lance, where the protective region protects the biological material during delivery into a biological structure.

### PRIORITY DATA

This application claims the benefit of U.S. Provisional Patent Application Ser. No. 61/487,535, filed on May 18, 2011, which is incorporated herein by reference.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

### SUMMARY OF THE INVENTION

The present disclosure provides systems, devices, and methods for protecting a biological material during delivery into a biological structure. In one aspect, for example, a device for protecting and delivering a preselected biological material into a biological structure can include a lance operable to maintain a charge capable of associating a biological material thereto and at least one protective region formed on or in the lance, where the protective region protects the biological material during delivery into a biological structure.

In another aspect, a method of protecting a biological material during delivery into a biological structure can include bringing into proximity outside of the biological structure a lance having at least one protective region and a preselected biological material, charging the lance with a polarity and a charge sufficient to electrically associate at least a portion of the biological material with the at least one protective region, and inserting the lance into the biological structure while the biological material is protected from damage and/or dislodgement by the at least one protective region. The method can also include discharging the lance to release at least a portion of the biological material from the at least one protective region of the lance and withdrawing the lance from the biological structure.

### DEFINITIONS OF TERMS

In describing and claiming the present invention, the following terminology will be used in accordance with the definitions set forth below.

The singular forms “a,” “an,” and, “the” include plural referents unless the context clearly dictates otherwise. Thus, for example, reference to “a support” can include reference to one or more of such supports, and reference to “an oocyte” can include reference to one or more of such oocytes.

As used herein, the term “biological material” can refer to any material that has a biological use and can be delivered into a biological such as a cell or a cell organelle. As such, “biological material” can refer to materials that may or may not have a biological origin. Thus, such material can include natural and synthetic materials, as well as chemical compounds, dyes, and the like.

As used herein, the term “charged biological material” may be used to refer to any biological material that is capable of being attracted to or associated with an electrically charged structure. Accordingly, charged biological material may be used to refer to those molecules having a net charge, as well as those molecules that have a net neutral charge but possess a charge distribution that allows attraction to the structure.

As used herein, the term “peptide” may be used to refer to a natural or synthetic molecule comprising two or more amino acids linked by the carboxyl group of one amino acid to the alpha amino group of another. A peptide of the present invention is not limited by length, and thus “peptide” can include polypeptides and proteins.

As used herein, the term “uncharged” when used in reference to a lance may be used to refer to the relative level of charge in the lance as compared to a charged biological material. In other words, a lance may be considered to be “uncharged” as long as the amount of charge on the needle structure is insufficient to associate a useable portion of a charged biological material. Naturally what is a useable portion may vary depending on the intended use of the biological material, and it should be understood that one of ordinary skill in the art would be aware of what a useable portion is given such an intended use. Additionally it should be noted that a lance with no measurable charge would be considered “uncharged” according to the present definition.

As used herein, the term “sample” when used in reference to a sample of a biological material may be used to refer to a portion of biological material that has been purposefully attracted to or associated with the lance. For example, a sample of a biological material such as DNA that is described as being associated with a lance would include DNA that has been purposefully attracted thereto, but would not include DNA that is attracted thereto through the mere exposure of the lance to the environment. One example of DNA that would not be considered to be a “sample” includes airborne DNA fragments that may associate with the lance following exposure to the air.

As used herein, “associate” is used to describe biological material that is in electrostatic contact with a structure due to attraction of opposite charges. For example, DNA that has been attracted to a structure by a positive charge is said to be associated or electrically associated with the structure.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### BRIEF DESCRIPTION OF THE DRAWINGS

FIG. 1A shows a schematic representation of a step of the delivery of a biological material into a cell in accordance with one embodiment of the present disclosure.

FIG. 1B shows a schematic representation of a step of the delivery of a biological material into a cell in accordance with another embodiment of the present disclosure.

FIG. 1C shows a schematic representation of a step of the delivery of a biological material into a cell in accordance with another embodiment of the present disclosure.

FIG. 1D shows a schematic representation of a step of the delivery of a biological material into a cell in accordance with another embodiment of the present disclosure.

FIG. 1E shows a schematic representation of a step of the delivery of a biological material into a cell in accordance with another embodiment of the present disclosure.

FIG. 1F shows a schematic representation of a step of the delivery of a biological material into a cell in accordance with another embodiment of the present disclosure.

FIG. 2 includes a graphical representation of a lance entering a cell in accordance with another embodiment of the present invention.

FIG. 3 includes an SEM image of a lance in accordance with another embodiment of the present invention.

FIG. 4 includes a cross-sectional view through a midline of a lance in accordance with another embodiment of the present invention.

FIG. 5 includes a cross-sectional view through a midline of a lance in accordance with another embodiment of the present invention.

FIG. 6 includes a cross-sectional view through a midline of a lance in accordance with another embodiment of the present invention.

FIG. 7 includes a cross-sectional view through a midline of a lance in accordance with another embodiment of the present invention.

### DETAILED DESCRIPTION

The present disclosure provides methods, devices, and associated systems for protecting a biological material during delivery into a cell. Also provided are methods, devices, and associated systems for maximizing the amount of biological material that can be delivered into a cell. Various conventional techniques have been used to introduce a biological material into a cell, and in some cases the biological material can become damaged or disassociated from the device during delivery. This can be problematic for techniques utilizing devices whereby the biological material is associated with an external surface of the delivery instrument, such as a lance.

Generally, the present methods and systems utilize the electrical association and dissociation of a biological material to a lance or other delivery device as a mechanism for delivering the biological material into a biological structure, such as a cell, cellular organelle, or any other structure of a biological origin. Because the biological material can be loaded onto the lance and subsequently released via changes in the charge state of the lance, internal microinjection channels are not required for the delivery of the biological material. As such, a lance can be smaller in size and can be formed in configurations that may not be possible with prior delivery devices. These delivery devices can have an outer shape and cross-section that is significantly smaller than traditional injection pipettes. Such smaller outer shapes may be less disruptive to cellular structures, and thus may allow delivery of the biological with less cellular damage.

Once a biological material has been electrically associated with a tip portion of the lance, the lance can be inserted through an outer portion of the biological structure. With a tip portion of the lance located within the biological structure, the lance can be discharged to release at least a portion of the biological material. Once the biological material has been delivered, the lance can be withdrawn.

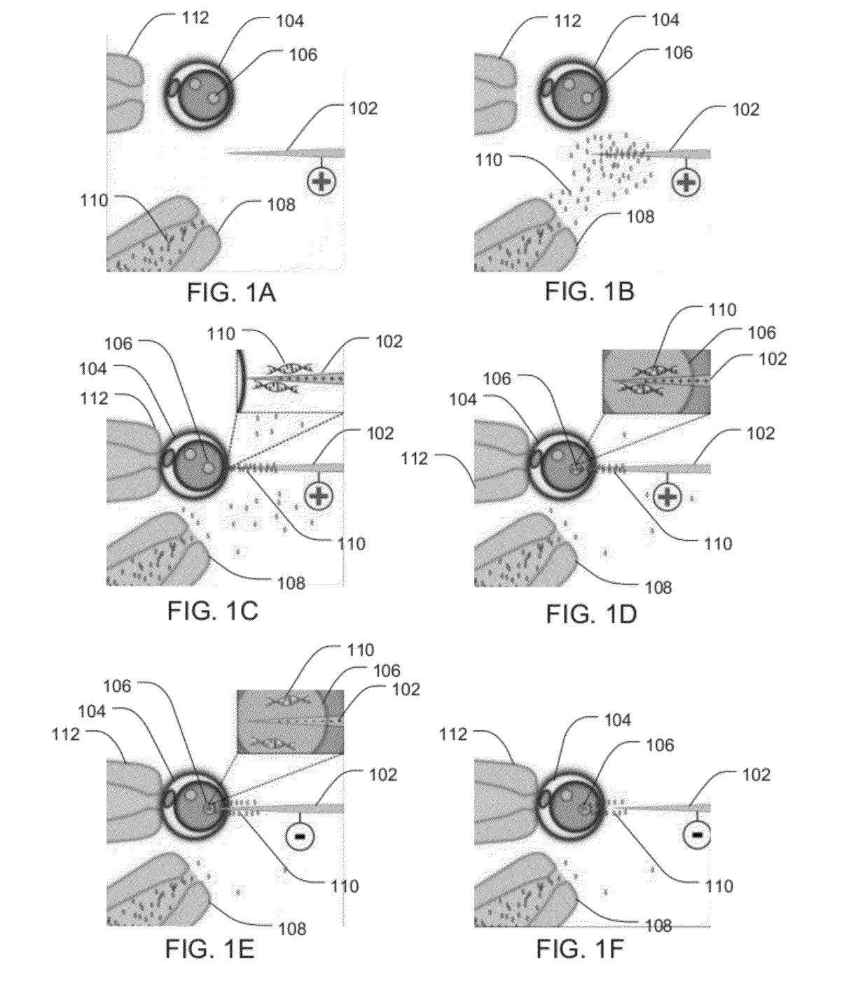

In one aspect, FIGS. 1A-F show exemplary sequences of steps that can be performed to introduce biological material into a biological structure. For this particular example, DNA is used as the biological material and a zygote is used as the biological structure. This example is intended to be non-limiting, and the description can generally be applied to other biological materials, cells, organelles, and the like. Electrically-mediated delivery of DNA into a biological structure can be accomplished due to the unequal charge distributions within DNA molecules. With an effective charge of 2 electrons per base pair, DNA can be manipulated by an electric field. FIG. 1A shows a lance 102 in proximity to a zygote 104 having a pronucleus 106. A biological material delivery device 108 containing the biological material 110 (e.g. DNA in this case), is positioned in proximity to the tip portion of the lance 102. The biological material delivery device 108 is shown as a micropipette, however any device capable of delivery a biological material to the tip portion of the lance is considered to be within the present scope. A cell manipulation device 112 is shown positioned in proximity to the zygote 104 to allow manipulation and/or securing of the cell during a biological material delivery procedure.

As is shown in FIG. 1A, the lance 102 is charged with a polarity and a charge sufficient to electrically associate the biological material 110 with a tip portion of the lance 102. In this case, the lance is positively charged in preparation for the accumulation of DNA at a tip portion of the lance. A return electrode can be placed in electrical contact with the medium surrounding the lance in order to complete an electrical circuit with the charging device and the lance (not shown). The lance is charged to a degree that is sufficient to associate DNA to the lance during the injection procedure. The amount of voltage sufficient to charge the lance can vary depending on a variety of factors, such as the desired speed of the loading of DNA on the lance, the composition of the lance material, the electrochemical nature of the medium surrounding the lance, and the like.

It should be noted, that various materials begin to decompose (e.g. by electrolysis) at voltages above a certain threshold voltage referred to as the decomposition voltage. The decomposition voltage can be different for different materials. In some cases, such decomposition can generate oxygen and hydrogen at the positively charged lance and the negatively charged return electrode, respectively. These electrolysis products can cause damage to the lance and negatively affect the cell being injected. As such, in one aspect the voltage that can be used to charge the lance can be at or below the decomposition voltage. In one specific aspect, the lance is charged with a voltage from about 1 V below the decomposition voltage to about the decomposition voltage. In another aspect, the lance is charged with a voltage from about 2 V below the decomposition voltage to about the decomposition voltage.

FIG. 1B shows the biological material 110 being released from the biological material delivery device 108. The biological material 110 can thus be released in the proximity of the tip portion of the lance 102 to effectively allow the biological material to associate with the tip portion of the lance. The polarity of the charge on the lance can depend on the charge distribution of the biological material. In the example shown in FIG. 1B, DNA is the biological material and therefore the lance is charged with a positive polarity to associate the DNA molecules thereto. The positive charge on the lance thus causes the negatively charged DNA to associate with and accumulate at the tip portion of the lance. If a biological material having a positive charge distribution is to be delivered, the lance can correspondingly be charged with a negative polarity in order to associate this positively charged biological material to the tip portion of the lance.

FIG. 1C shows the zygote 104 secured by a cell manipulation device 112. The cell can be manipulated, secured, and/or held in position by a variety of mechanisms. It should be noted that any technique, device, or system for manipulating, securing, and/or holding a cell in position is considered to be within the present scope. In one aspect, for example, the cell can be held in position by a suction pipette, as is shown in FIG. 1C (cell manipulation device 112). A slight suction at the end of such a pipette can hold a cell for sufficient time to accomplish a biological material delivery procedure into the cell.

Once the zygote 104 is restrained, the lance 102 can be oriented into a position relative to the zygote into which the biological material will be introduced, as is shown in FIG. 1C. The upper right inset of FIG. 1C shows a close up view of the tip portion of the lance 102 having the biological material 110, in this case DNA, associated therewith. As has been noted, a biological structure can also include a cellular organelle. FIG. 1C shows the lance 102 oriented into a position that is aligned with an organelle 106 of interest.

As is shown in FIG. 1D, following positioning, the lance 102 penetrates an outer portion of the zygote 104 and is directed and inserted into an organelle 106. The zygote 104 can be held in position by the cell manipulation device 112 during the injection procedure to minimize movement of the zygote. The minimization of movement of the zygote can facilitate the insertion of the lance into the organelle, particularly for small organelles such as pronuclei, while also potentially reducing movement-induced damage of the cell.

As is shown in the upper right inset of FIG. 1D, the biological material 110 that is associated with the tip portion of the lance 102 is carried into the organelle 106 along with the lance. In this example, DNA associated with the tip portion of the lance is carried into the pronucleus of the cell along with the lance. In one aspect, the lance is inserted into the zygote and into the organelle in a reciprocating motion along an elongate axis of the lance. Such an insertion method may minimize tearing of the cell membrane and internal damage to the zygote. Once the lance 102 is inserted into the zygote 104, the lance 102 is discharged to allow the release of at least a portion of the biological material 110 from the tip portion of the lance 102, thus delivering the biological material 110 to the zygote 104 as is shown in FIG. 1E. The upper right inset of FIG. 1E shows the biological material 110 being dissociated from the lance 102 in the organelle 106. Discharging the lance to release the biological material can be accomplished in a variety of ways. In one aspect, for example, discharging the lance can include decreasing the charge on the lance to a degree that is sufficient to release at least a portion of the biological material from the lance. In another aspect, discharging the lance can include releasing the charge on the lance sufficient to release the biological material or at least a portion of the biological material from the lance. In yet another aspect, discharging the lance can include reversing the polarity of the charge on the lance to release at least a portion of the biological material. Such a reversal charges the lance to a polarity that is opposite from the polarity used to attract the biological material (e.g. the DNA) to the tip portion of the lance. Thus, a positively charged lance can be reversed to a negative charge to cause a negatively charged biological material such as DNA to be released from the surface of the lance. Thus, depending on the manner in which the lance is discharged, from only a portion to substantially all of the biological material associated with the lance can be released.

Following release of the biological material, the lance 102 can be withdrawn from the zygote 104 as is shown in FIG. 1F. The biological material 110 delivered can thus remain in the zygote following withdrawal of the lance 102. Once the lance 102 is withdrawn, the cell 104 can be released from the cell manipulation device 112.

When a lance having biological material associated with its surface is inserted through a biological structure such as, for example, a cell membrane, the biological material can become damaged and/or displaced or otherwise disassociated from the lance due to the movement of the biological structure relative to the surface of the lance. Such disruptive effects can decrease the quantity of, and possibly the quality of, biological material that is actually delivered into the biological structure. In addition to external biological structures such as cell walls or cell membranes, internal biological structures can cause similar disruptive effects, thus possibly limiting the biological material arriving at a target location within the biological structure.

Accordingly, methods, systems, and devices for protecting biological material during delivery into a biological structure are provided. In one aspect, for example, a method can include bringing into proximity outside of a biological structure a lance having at least one protective region and a preselected biological material. The lance is charged with a polarity and a charge sufficient to electrically associate at least a portion of the biological material at least partially within the at least one protective region. The lance is then inserted into the biological structure while the biological material is protected from damage and/or displacement by the at least one protective region. Following insertion into the biological structure, the lance is discharged to release at least a portion of the biological material from the at least one protective region of the lance. Releasing the biological material from the protective region thus delivers the biological material into a location within the cell. The lance can then be withdrawn from the biological structure.

It should be noted that in some aspects the protective region may provide sufficient protection that the biological material may remain in the protective region during lance insertion into the cell. For example, in one aspect the lance can be charged to associate the biological material within the protective region. The lance can be discharged and inserted into the cell, while the biological material remains associated with the protective region. Once in the cell, the lance can be oppositely charged to disassociate the biological material from the protective region.

In another aspect, a device for protecting and delivering a preselected biological material into a biological structure is provided including a lance that is operable to maintain a charge capable of associating a biological material thereto, and at least one protective region formed on or in the lance. The protective region(s) is operable to protect the biological material during delivery into a biological structure.

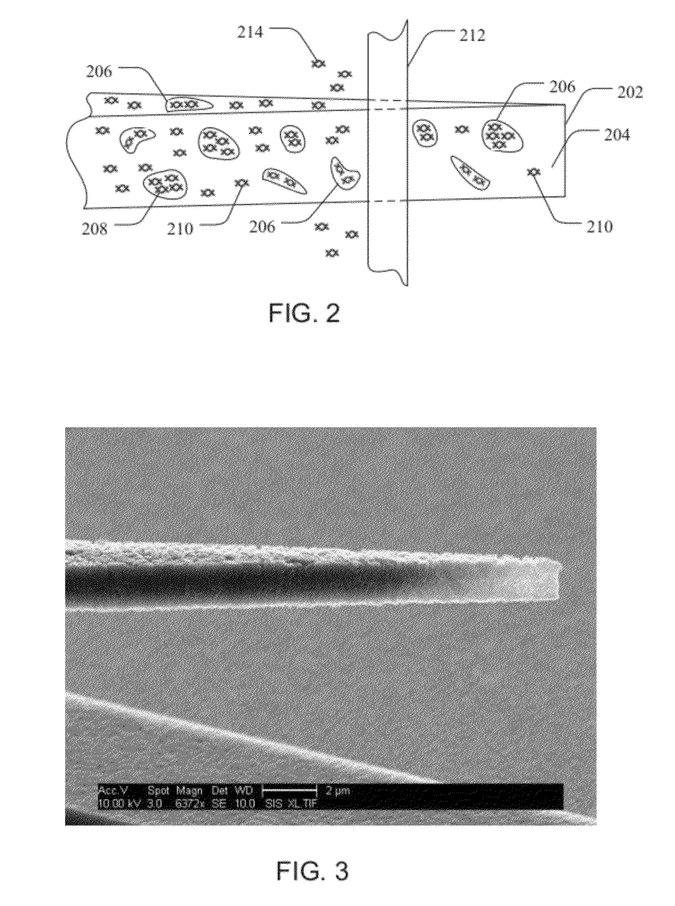

Turning to FIG. 2, a lance 202 is shown having an outer surface 204 with a plurality of protective regions 206 located therein. The lance is shown with associated protected biological material 208 located within the protective regions 206 and with associated unprotected biological material 210 located across the outer surface 204 of the lance 202 that are not located within the protective regions. The lance 202 is shown being inserted through a biological structure 212, which causes the dislodgement of biological material from the outer surface 204 of the lance 202. Such disassociated biological material is shown at 214. Thus, inserting the lance through the biological structure can cause at least a portion of the unprotected biological material to be disassociated and/or damaged prior to entry into the cell. The protected biological material located within the protective regions is at least partially shielded from the disruptive influence of the biological structure as it slides across the outer surface of the lance. It is also contemplated that the protected biological material may be at least partially shielded from other potentially disruptive influences such as, for example, fluid currents, viscosity changes, pressure differentials, voltage potential fluctuations, and the like.

Various sizes and configurations of protective regions are contemplated, and any such size and/or configuration capable of providing protection to biological material during delivery is considered to be within the present scope. In one aspect, for example, the protective regions can be of a size sufficient to protect a biological material from damage and/or disassociation from the lance. As such, the size of the protective region can vary depending on the biological material being associated therewith. For example, small molecules can be accommodated in smaller protective regions as compared to large macromolecules such as DNA. That being said, in one aspect protective regions can be from about 5 nm to about 1 micron in size. In another aspect, protective regions can be from about 10 nm to about 500 nm in size. In yet another aspect, protective regions can be from about 50 nm to about 250 nm in size. In another aspect, the protective regions can sufficiently large to protect a biological material from damage and/or disassociation from the lance and sufficiently small to minimize snagging on membranous structures such as the cellular membrane. It should be noted that, when referring to a size of the protective regions, measurements can be the approximate size of openings or depressions in the lance surface, the approximate size of the protective region within the lance body, the approximate surface area of the protected region, or the like.

It should be noted that it is not necessarily the size of a protective region that may cause damage to cellular structures during delivery, but rather the size with respect to the location and configuration on the lance. For example, a 1 μm protrusion from the side of lance may cause significantly more damage to the cell than a 2 μm hollow region extending into the lance with little or no protrusion. Furthermore, the size of a protrusion from a protective region that results in undesirable damage to a biological structure can vary depending on the cell. Such protrusions can be larger in big cells such as zygotes as compared to small cells such as neurons. That being said, in one aspect the portion of a protective region that causes disruption to a biological structure can be less than or equal to about 50 nm. In another aspect, the portion of a protective region that causes disruption to a cellular structure can be less than or equal to about 10 nm. In yet another aspect, the portion of a protective region that causes disruption to a cellular structure can be less than or equal to about 1 nm.

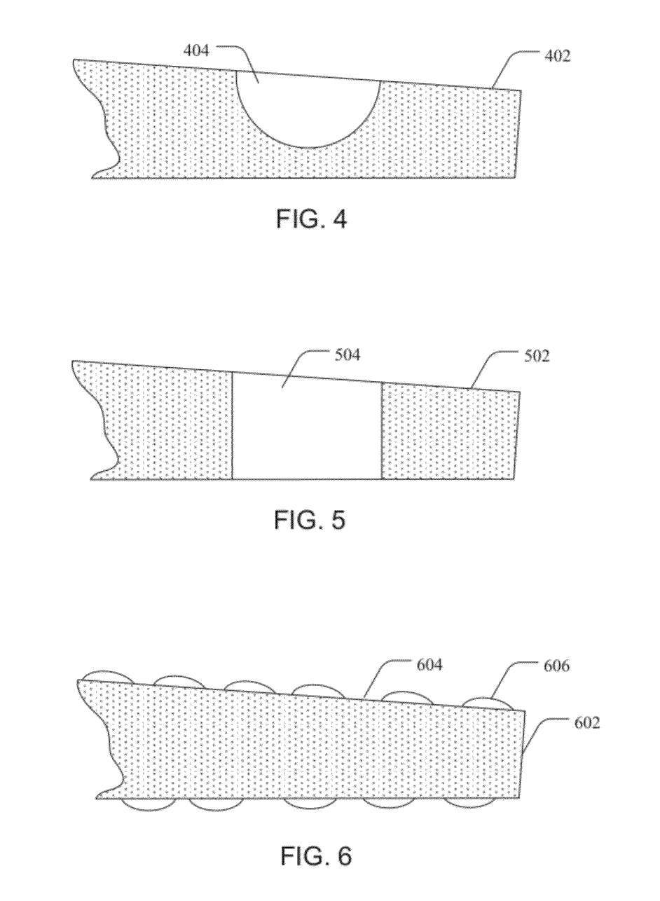

One exemplary technique for creating a lance having an outer surface covered with protective regions is to fabricate the lance from a polycrystalline material through a micromachining process. Randomly oriented crystallographic grains of the polycrystalline material can cause surface depressions at the grain boundaries between the crystals. These surface depressions can be of a size sufficient to function as protective regions for the biological material. An SEM image of one example of a polycrystalline silicon lance is shown in FIG. 3. The lance shows an overall smooth gross surface morphology with nano-scaled features (i.e. indentations, disruptions, etc.) that are capable of protecting biological material associated therewith. In one aspect, the surface depressions can be enhanced (i.e. increased in size, surface area, depth, etc.) by chemical etching of the grain boundaries between the crystals.

In other aspects, a lance can contain larger protective regions for protecting biological material during delivery. In one aspect, as is shown in FIG. 4, a lance 402 can include at least one protective region 404 shaped as a depression extending partially through the body of the lance. When the lance is charged, biological material that is attracted into the protective region 404 can be protected during delivery into a biological structure. While the protective region 404 of FIG. 4 is shown as a hemisphere, such a depression can be of any useful shape. Non-limiting examples include cylinders, ovals, squares, rectangles, polygons, slots, grooves, triangles, and the like, including combinations thereof. Furthermore, in some aspects the protective regions can be the pores of a porous material used to make the lance.

In addition to protective regions extending partially through the body of the lance, in some aspects a protective region can extend entirely through the body of the lance. As is shown in FIG. 5, for example, a lance 502 can include a protective region 504 that extends entirely through the body of the lance 502. It should be noted that, for protective regions such as those shown in FIGS. 4 and 5, the edges around the protective region can be rounded or smoothed in order to reduce snagging or other damage to cellular structures.

In another aspect, as is shown in FIG. 6, a lance 602 can include one or more protective regions 604 that are formed by depositing a protective material 606 onto one or more surfaces of the lance. As such, the protective material can deflect portions of the biological structure during delivery to provide protection to biological material associated with spaces in between the deposited protective material. The protective material can be applied to the lance by any known deposition process, including CVD, PVD, electrodeposition, and the like. The protective material can be deposited in a uniform or in a non-uniform pattern. In this manner, the protective region is formed as a protrusion extending from the surface of the lance.

As has been described, the size and configuration of the protective region can vary depending on the design of the lance, the lance material, and the nature of the delivery procedure, including various user preferences. For example, in one aspect a protective region can be up to a size or volume that maintains sufficient material in the lance to provide adequate cross sectional strength during delivery of the biological material. In another aspect, multiple protective regions can be up to a collective size or volume that maintains sufficient material in the lance to provide adequate cross sectional strength during delivery of the biological material.

In addition to the micromachining of the polycrystalline lance example described above, various techniques are contemplated for forming protective regions in the lance surface, and any such technique is considered to be within the present scope. For example, such techniques can include MEMS fabrication, etch processing, micromachining, laser ablation, physical abrasion, and the like, including combinations thereof. Additionally, materials having a structure that generates protective regions during manufacture of the lance, such as porous materials, can also be used.

Additionally, the protective regions can provide additional benefits to the lance. For example, one issue that may arise as a lance decreases in size relates to the surface area that is available for association with a biological material. A smaller lance tip has a smaller surface area for holding a charge as compared to a larger lance tip. This smaller surface area can limit the amount of biological material that can be associated therewith. By introducing protective regions into the lance, the surface area of the lance tip is increased, thereby increasing the amount of biological material that can be associated with the charged lance tip. Accordingly, in one aspect protective regions can be provided in a lance to increase the surface area of the lance.

As has been described, a charge is introduced into and held by the lance in order to electrically associate the biological material to the lance. Various lance materials are contemplated for use in constructing the lance, and any material that can be formed into a lance structure and is capable of carrying a charge is considered to be within the present scope. Non-limiting examples of lance materials can include a metal or metal alloy, a conductive glass, a polymeric material, a semiconductor material, carbon nanotube, and the like, including combinations thereof. In one aspect, a lance can be a carbon nanotube filled with a material such as carbon, silicon, and the like. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof.

Non-limiting examples of useful semiconductor materials can include germanium, gallium arsenide, and silicon, including various forms of silicon such as amorphous silicon, monocrystalline silicon, polycrystalline silicon, and the like, including combinations thereof. Indium-tin oxide is a material that is also contemplated for use as a lance material. Additionally, in some aspects the lance can be a conductive material that is coated on a second material, where the second material provides the physical structure of the lance. Examples can include metal-coated glass or metal-coated quartz lances. The lance can also include a hollow, non-conductive material, such as a glass, where the hollow material is filled with a conductive material. Depending on the design, the lance can be manufactured using various techniques such as wire pulling, chemical etching, MEMs processing, and the like.

Any size and/or shape of lance capable of delivering biological material into a cell is considered to be within the present scope. The size and shape of the lance can also vary depending on the cell receiving the biological material. The effective diameter of the lance, for example, can be sized to maximize survivability of the cell. It should be noted that the term “diameter” is used loosely, as in some cases the cross section of the lance may not be circular. Limits on the minimum diameter of the lance can, in some cases, be a factor of the material from which the lance is made and the manufacturing process used. In one aspect, for example, the lance can have a tip diameter of from about 5 nm to about 3 microns. In another aspect, the lance can have a tip diameter of from about 10 nm to about 2 microns. In another aspect, the lance can have a tip diameter of from about 30 nm to about 1 micron. In a further aspect, the lance can have a tip diameter that is less than or equal to 1 micron. As such, in many cases the tip diameter of the lance can be smaller than the resolving power of current optical microscopes, which is approximately 1 micron. As is noted above, lance tips are contemplated that can have cross sections that are not circular. In such cases, it is intended that the circumference of a circle defined by the tip diameters disclosed above would be substantially the same as an outer circumferential measurement of a non-circular lance tip.

The length of the lance can be variable depending on the design and desired attachment of the lance to the lance manipulation system. Also, the portion of the lance that is contacting and/or passing through a portion of the cell can vary in length depending on the lance design and the depth of the area into which the biological material is to be delivered. For example, delivering biological material to an area located near the surface of a cell can be accomplished using a shorter lance as compared to delivery to an area located deep within a cell. This would not preclude, however, the use of longer lances for delivery into areas near the cellular surface. For example, a relatively long lance may be used to deliver biological material in an application where only a small portion (e.g., only the tip) of the lance penetrates a cell. It should be noted that the lance length can be tailored to the delivery situation and to the preference of the individual performing the delivery.

Thus the length of the lance can be any length useful for a given delivery operation. For example, in some aspects, the lance can be up to many centimeters in length. In other aspects, the lance can be from a millimeter to a centimeter in length. In another aspect, the lance can be from a micron to a millimeter in length. In one specific aspect, the lance can be from about 2 microns to about 500 microns in length. In another specific aspect, the lance can be from about 2 microns to about 200 microns in length. In yet another specific aspect, the lance can be from about 10 microns to about 75 microns in length. In a further specific aspect, the lance can be from about 40 microns to about 60 microns in length.

Additionally, the shape of the lance, at least through the portion of the lance contacting the cell, can vary depending on the design of the lance and the depth to which the biological material is to be injected into the cell. A high lance taper, for example, may be more disruptive to cellular membranes and internal cellular structures than a low taper. In one aspect, for example, the lance can have a taper of from about 1% to about 10%. In another aspect, the taper can be from about 2% to about 6%. In yet another aspect, the taper can be about 3%. The taper of the lance can also be described in terms of the size of the disruption in the cell membrane following insertion. In one aspect, for example, the approximate diameter of the disrupted area of the cell membrane following lance insertion is from about 0.5 nanometers to about 8 microns. In another aspect, the approximate diameter of the disrupted area of the cell membrane following lance insertion is from about 2 micron to about 5 microns.

The overall shape and size of the lance can also be designed to take into account various factors, including those involved with the delivery procedure, as well as the materials utilized to make the lance. For example, in one aspect a lance can be designed having sufficient cross sectional strength to allow biological material delivery, while at the same time minimizing the damage done to the biological structure from the lance's cross sectional area. As another example, the lance can be designed to have a cross sectional area sufficient to minimize damage to the biological structure, while at the same having sufficient surface area to which biological material can be associated.

Different materials can also affect the design of the size and shape of the lance. Some materials may not hold a charge sufficient to associate the biological material to the lance tip at smaller sizes. In such cases, larger size lances can be used to facilitate a higher charge capacity. It may be difficult to form particular sizes and shapes of the lance from certain materials. In such cases, the lance size and shape can be designed to the properties of the desired material. For example, a material such as gold may not be capable of supporting the lance tip at very small diameters due to inadequate strength at smaller sizes, or it may not be possible or feasible to create a very small diameter tip with gold. If the use of a gold lance is desired, the lance size and shape can thus be designed with the properties of gold in mind.

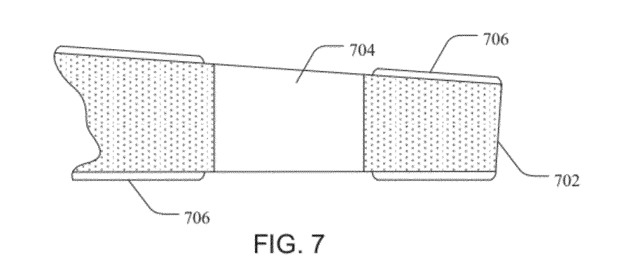

In another aspect, lances in accordance with the present disclosure can be constructed so as to facilitate the electrical association of biological material within the protective region or protective regions. In some aspects, biological material can be associated within the protective region while minimizing the association of biological material elsewhere on or in the lance. As is shown in FIG. 7, for example, a lance 702 having a protective region 704 can also include a passivation material 706 applied to an outer surface of the lance. Passivation can be used to focus current flow through a protective region, thus potentially causing the biological material to preferentially associate in the protective region. The passivation material can be any material that is less conductive than the material of the lance, and can thus focus current flow through the protective region. Non-limiting examples of such materials can include oxides, nitrides, oxynitrides, ceramics, polymers, and the like, including combinations thereof.

Biological material can be delivered into a variety of biological structures, including various cell types. Both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature. Additionally, various types of biological materials are contemplated for delivery into a biological structure, and any type of biological material that can be electrostatically delivered is considered to be within the present scope. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, and the like, including combinations thereof. In one aspect, the biological material can include DNA, cDNA, RNA, siRNA, tRNA, mRNA, microRNA, and combinations thereof. In another aspect, the biological material can include DNA and/or cDNA.

A charging system used to charge the lance can include any system capable of electrically charging, maintaining the charge, and subsequently discharging the lance. Non-limiting examples can include batteries, DC power supplies, photovoltaic cells, static electricity generators, capacitors, and the like. The charging system can include a switch for activation and deactivation, and in some aspects can also include a polarity switch to reverse polarity of the charge on the lance. In one aspect the system may additionally include multiple charging systems, one system for charging the lance with a charge, and another charging system for charging the lance with an opposite polarity charge. In one example scenario, an initially uncharged lance is brought into contact with a sample of a biological material. The biological material can be in water, saline, or any other liquid capable of maintaining biological material. A charge opposite in polarity to the biological material is applied to the lance, thus associating a portion of the biological material with the lance. The lance can then be moved into the biological structure, and lance can be discharged, thus releasing the biological material.

The lance can be manipulated by any mechanism capable of aligning and moving the lance. Such a lance manipulation system can include any system or device capable of orienting and moving a lance. Non-limiting examples of lance manipulation systems include mechanical systems, magnetic systems, piezoelectric systems, electrostatic systems, thermo-mechanical systems, pneumatic systems, hydraulic systems, and the like. In one aspect, the lance manipulation system can be one or more micromanipulators. The lance may also be moved manually by a user. For example, a user may push the lance along a track from first location to a second location.

In one aspect, the lance can be moved by the lance manipulation system in a reciprocal motion along an elongate axis of the lance. In other words, the lance can move forward into a biological structure and backward out of the biological structure along the same path. By moving along the elongate axis of the lance, the minimum cross sectional area of the lance is driven through biological structure such as a cell membrane and/or an organelle of interest. This minimal cross sectional exposure can limit the cellular disruption, thus potentially increasing the success of the biological material delivery procedure.

For cellular delivery, the cell can be manipulated and or held in position by a variety of mechanisms. It should be noted that any technique, device, or system for manipulating and/or holding a cell in position is considered to be within the present scope. In one aspect, for example, the cell can be held in position by a suction pipette. A slight suction at the end of such a pipette can hold a cell for sufficient time to accomplish a biological material delivery procedure into the cell. Additionally, supporting arms or other physically restraining structures can be used to hold the cell in position during the delivery procedure. Various configurations for support structures would be readily apparent to one of ordinary skill in the art once in possession of the present disclosure, and such configurations are considered to be within the present scope.

Further exemplary details regarding lances, charging systems, lance manipulation systems, and cellular restraining systems can be found in U.S. patent application Ser. Nos. 12/668,369, filed Sep. 2, 2010; 12/816,183; filed Jun. 15, 2010; 61/380,612, filed Sep. 7, 2010; and 61/479,777, filed on Apr. 27, 2011, each of which is incorporated herein by reference.

It should be noted that the design of a system for delivering biological material into a biological structure can vary due to the interdependencies of various system parameters. Combinations of features can thus influence other features, both in terms of system design and in terms of system use. Features can thus be mixed and matched to create a delivery system for a given purpose or desirable performance. For example, the materials and configuration chosen for the lance may have properties allowing a greater or lesser charge capacity, thus influencing the voltage, current, and electrical timing of the charging and discharging. A smaller tip diameter can more effectively enter a biological structure with potentially less damage, but may have a smaller surface area for the association of biological material. The association capacity of the lance for biological material can thus be increased, for example, by utilizing lance materials capable of holding a higher relative charge, or by utilizing a non-circular shape for the lance tip that increases surface area while minimizing the penetration damage of the lance. Thus, if a particular feature is desired for a lance, other features can be varied to accommodate such a design. As such, it should be understood that the various details described herein should not be seen as limiting, particularly those involving dimensions or values. It is contemplated that a wide variety of design choices are possible, and each are considered to be within the present scope.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A device for protecting and delivering a preselected biological material into a biological structure, comprising:
a lance operable to maintain a charge capable of associating a biological material thereto; and
at least one protective region formed on or in the lance, the at least one protective region being operable to protect the biological material during delivery into a biological structure.

2. The device of claim 1, wherein the at least one protective region is a plurality of protective regions.

3. The device of claim 1, wherein the lance is a polycrystalline material and the at least one protective region includes surface depressions at grain boundaries between crystals in the polycrystalline material.

4. The device of claim 3, wherein the surface depressions have been enhanced by chemical etching of the grain boundaries.

5. The device of claim 1, wherein the at least one protective region includes at least one surface depression formed in the lance.

6. The device of claim 5, wherein the at least one surface depression is formed by a technique selected from the group consisting of MEMS fabrication, etch processing, micromachining, laser ablation, physical abrasion, and combinations thereof.

7. The device of claim 1, wherein the at least one protective region is formed by deposition of a protective material on the lance.

8. The device of claim 1, wherein the at least one protective region is a hollow portion of the lance.

9. The device of claim 1, further comprising a passivation layer deposited on the lance to focus association of the biological material at the at least one protective region.

10. A method of protecting a biological material during delivery into a biological structure, comprising:
bringing into proximity outside of the biological structure: i) a lance having at least one protective region; and ii) a preselected biological material;
charging the lance with a polarity and a charge sufficient to electrically associate at least a portion of the biological material with the at least one protective region;
inserting the lance into the biological structure while the biological material is protected from damage and/or dislodgement by the at least one protective region;
discharging the lance to release at least a portion of the biological material from the at least one protective region of the lance; and
withdrawing the lance from the biological structure.

11. The method of claim 10, wherein the biological structure is a cell.

12. The method of claim 11, wherein the cell is a zygote.

13. The method of claim 10, wherein the biological structure is a cellular organelle.

14. The method of claim 10, wherein inserting and withdrawing the lance is performed with a reciprocating motion along an elongate axis of the lance.

15. The method of claim 10, wherein the biological material includes a member selected from the group consisting of DNA, RNA, peptides, polymers, organic molecules, inorganic molecules, ions, and combinations thereof.

16. The method of claim 10, wherein the biological material includes DNA.
